(19) United States
(12) Patent Application Publication
(10) Pub. No.: US 2013/0131640 A1
(43) Pub. Date: May 23, 2013

(54) METHOD OF CREATING AND SORTING FUSED CELLS

(71) Applicants: Thomas E. Wagner, Greenville, SC (US); Yanzhang Wei, Greer, SC (US); Jinhua Li Wei, Greer, SC (US)

(72) Inventors: Thomas E. Wagner, Greenville, SC (US); Yanzhang Wei, Greer, SC (US); Jinhua Li Wei, Greer, SC (US)

(73) Assignee: Orbis Health Solutions LLC

(21) Appl. No.: 13/697,166

(22) PCT Filed: May 9, 2011
(86) PCT No.: PCT/US11/35748

§ 371 Date: Jan. 16, 2013

Related U.S. Application Data
(60) Provisional application No. 61/333,061, filed on May 10, 2010.

(51) Int. Cl. C12N 5/16

(52) U.S. Cl. CPC C12N 5/16
USPC 604/522; 435/346; 435/450

(57) ABSTRACT

An efficient and simplified method for preparing and sorting fused cells is described herein. This approach yields fused cells useful in a variety of applications, including clinical treatment regimens, as cellular modulators of the immune system.

### BACKGROUND OF THE INVENTION

The present invention relates to hybrid cells, also known as fusion cells, and methods of making and using hybrid cells.

Hybrid cells can be generated through cell fusion between two or more of cells that can be of the same cell type or different cell types. Hybrid cells can be used in medical applications, such as for personalized immunotherapy in a clinical treatment setting.

A hybrid cell can be produced by fusing a dendritic cell (DC) and a tumor cell. DC is essentially the control center of the immune system and when this critical cell presents antigen epitopes generated from proteins within its cytoplasm, naïve CD8 T-cells are activated, initiating the process to generate antigen targeted cytotoxic T lymphocytes (CTLs) via the MHC class I pathway. CTLs are the principal weapon of the immune system to eliminate cellular disease and play an important role in immunotherapy.

Immunotherapy has continued to prove effective in the treatment of cancer from its historic beginnings to the present without significant adverse side effects. Unfortunately there are significant barriers to accomplishing this goal. For one, because immunotherapy depends upon the action of the patient's own immune system, it is personalized and requires effective production of hybrid cells. The cell sorting technology that is currently used to isolate the hybrid cells that make up the therapeutic vaccine is not readily available at most major hospitals or the typical clinics where many oncologists practice. Additionally, the inefficiency of the methodology in the art to create cell fusions means that a relatively large number of tumor cells and DCs must be harvested from patients in order to generate the vaccine.

Accordingly, an efficient, simplified, and automated system for the production and separation of fused cells, such as tumor/DC hybrid cell vaccines is needed, to make hybrid cell vaccine treatments available to the typical cancer patient. More generally, the art is in need of broadly applicable and rapid methods for preparing and isolating hybrid cells.

### SUMMARY OF THE INVENTION

It is, therefore, an object of the invention to provide solutions to the aforementioned deficiencies in the art.

In one embodiment of the invention is a method for separating a fused cell from a population of unfused cells comprising: (a) contacting a first cell or cells with a second cell or cells under conditions suitable for cell fusion, wherein the first cell or cells is/are linked to a member of a first specific binding pair and the second cell or cells is/are linked to a member of a second specific binding pair, and wherein the member of the first specific binding pair and the member of the second specific binding pair are different; (b) adding a carrier conjugated to a member of a specific binding pair complementary to the member of the first specific binding pair; (c) isolating cells linked to the member of the first specific binding pair based on properties of the carrier; (d) adding to the cells isolated in (c) a carrier conjugated to a member of a specific binding pair complementary to the member of the second specific binding pair; and (e) isolating cells linked to the member of the second specific binding pair based on properties of the carrier, wherein the fused cells are separated from the unfused cells.

Also described is a method for enhancing the rate of cell fusion between reactant cells comprising contacting a first reactant cell or cells and a second reactant cell or cells under conditions for cell fusion, wherein the first reactant cell or cells is/are linked to a member of a specific binding pair and the second reactant cell or cells is/are linked to a complementary member of the specific binding pair.

The fused cell can comprise a cell of a first cell type and a cell of a second cell type, such as a dendritic cell and a tumor cell.

### BRIEF DESCRIPTION OF THE DRAWINGS

FIG. 1 illustrates the strategy of preparing and isolating a hybrid cell.

FIG. 2 is a FACS (fluorescence-activated cell sorting) detection curve demonstrating the binding and cleavage between poly(U) beads and oligo(A) linked biotin/streptavidin.

FIG. 3 is a picture depicting isolated hybrid tumor/DC cells. Tumor cells are indicated by arrows.

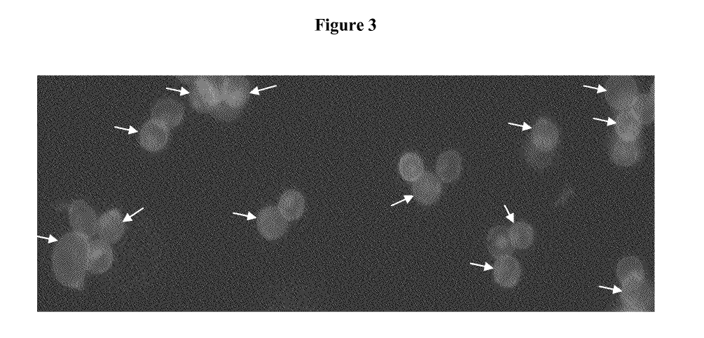

FIG. 4 is a picture depicting DCs (small circles), and hybrid tumor/DC cells (larger circles). Some of the hybrid cells are indicated by arrows.

### DETAILED DESCRIPTION OF THE INVENTION

In one aspect, the present invention provides a rapid and efficient method of preparing and isolating fused cells that are useful in a variety of clinical and non-clinical applications. For example,

### Definitions

Specific Binding Pair: A “specific binding pair” as described herein connotes a pair of molecules (each being a member of a specific binding pair) which are naturally derived or synthetically produced. One member of the pair of molecules specifically binds, either covalently or non-covalently, to the other member of the specific binding pair and is therefore defined as complementary with a particular spatial and polar organization of the other molecule. Examples of types of specific binding pairs are oxyamine/aldehyde, azide acetylide, and biotin-avidin, or other bioorthogonal agents that are non-toxic and non-interacting with biological functionality while proceeding under physiological conditions. A specific binding pair, for the purpose of this disclosure, does not include biological molecules such as antigens/antibody binding pairs or ligand/receptor binding pairs that interfere with the function of the cell.

Hybrid Cell: A “hybrid cell” as described herein connotes a fused cell comprising a tumor cell and an antigen presenting cell, such as a dendritic cell or monocyte.

Carrier: A “carrier” as described herein connotes an object having a specific physical or chemical characteristic, such as size, shape, weight, color, affinity, or a magnetic or electric property, that enables its separation from other objects. For example, the carrier can enable cell sorting based on density, size, magnetic character, charge, etc.

Method of Sorting a Fused Cell

In one embodiment of the present invention is a method for separating a fused cell from a population of unfused cells. An exemplary fused cell is a hybrid cell (a tumor cell fused to an antigen presenting cell), which is particularly useful as a vaccine to stimulate the patient's own immune response and treat or prevent a disease such as cancer. Also contemplated herein is a fused cell that comprises a plasma cell and a cancer cell which, like conventional hybridomas, are useful in preparing monoclonal antibodies. In still another embodiment, the fused cell comprises an antigen presenting cell that lacks an accessory component needed for an immunogenic response and a cell from an organ destined for transplant in a patient. These cells may be used to induce tolerance to the transplant cells, thereby reducing the incidence of transplant rejection.

The inventors discovered that chemical moieties linked to N-hydroxysuccinimide (NHS) esters via stretches of polyethylene glycol (PEG) are uniquely able to selectively modify the external surface proteins of living cells under physiological conditions. Alternatively, chemical moieties linked to NHS esters and containing negative charge are also a suitable modification. Such modifications appear to have no deleterious effects on the viability or function of the modified cells. Accordingly, the inventors have taken advantage of this property and developed a method for sorting fused cells which can unexpectedly be done with greater ease and efficiency compared to prior art methods.

Thus, one embodiment of the present invention is a method of separating fused cells that is rapid, simple to use, and applicable to all types of cells. The inventive approach involves bringing at least two cells (reactant cells) into contact under conditions that promote cell fusion, and then purifying the resultant fused cell. The reactant cells may be two or more of the same type of cell or two or more cells of a different type. In this embodiment, a first cell is linked or attached to a member of a specific binding pair. The complementary member of the specific binding pair is attached to a carrier such as a magnetic bead or other particle of a particular size which will ultimately be used to identify and isolate the first cell. In other words, the first cell linked to the member of a specific binding pair can be separated from other cells in a cell mixture that are not linked to the member of a specific binding pair.

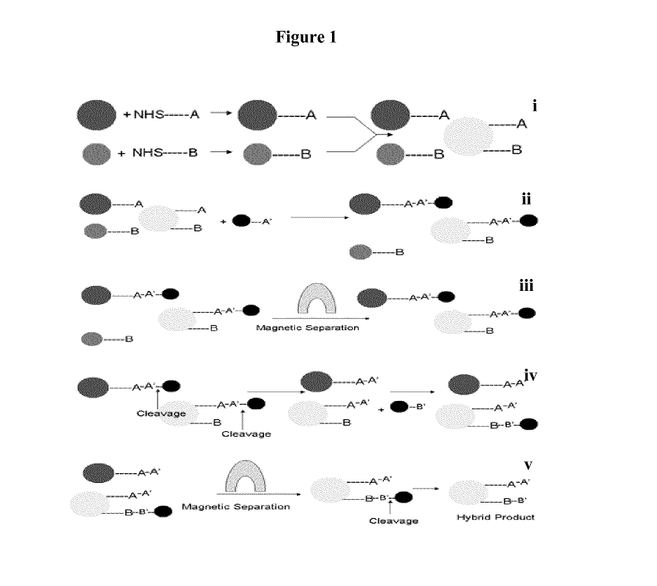

The general strategy of the inventive method is illustrated in FIG. 1. At step (i) in the Figure, a cell of type X linked to a first member of a specific binding pair is fused with a cell of type Y linked to a second member of a specific binding pair that is different from the first member of a specific binding pair. In the resulting cell mixture, there are fused cells and unfused cells of type X and type Y. At step (ii), cells, including the fused cells, come in contact with a complementary first member of a specific binding pair linked to a carrier such as a superparamagnetic microbead (SPM MB). Cells linked to the first member of a specific binding pair, including the fused cells and unfused cells of type X, bind the complementary first member of a specific binding pair. At step (iii), the cells are magnetically separated to remove cells that did not bind the complementary first member of a specific binding pair, e.g., unfused cells of type Y. At step (iv), cells that bound to the magnetic beads are released from the beads via cleavage between the complementary first member of a specific binding pair and the magnetic bead. The released cells are then contacted with a complementary second member of a specific binding pair conjugated to a carrier. At this point, only fused cells bind to the complementary second member of a specific binding pair, because unfused cells are not linked to the second member of a specific binding pair. At step (v), the fused cells are again magnetically separated from the cell mixture and subsequently released from the magnetic beads via a cleavage between the complementary second member of a specific binding pair and the carrier.

In one aspect, a specific binding pair is oxyamine/aldehyde or aldehyde/oxyamine. Oxyamine can form a highly selective linkage with aldehyde as shown below.

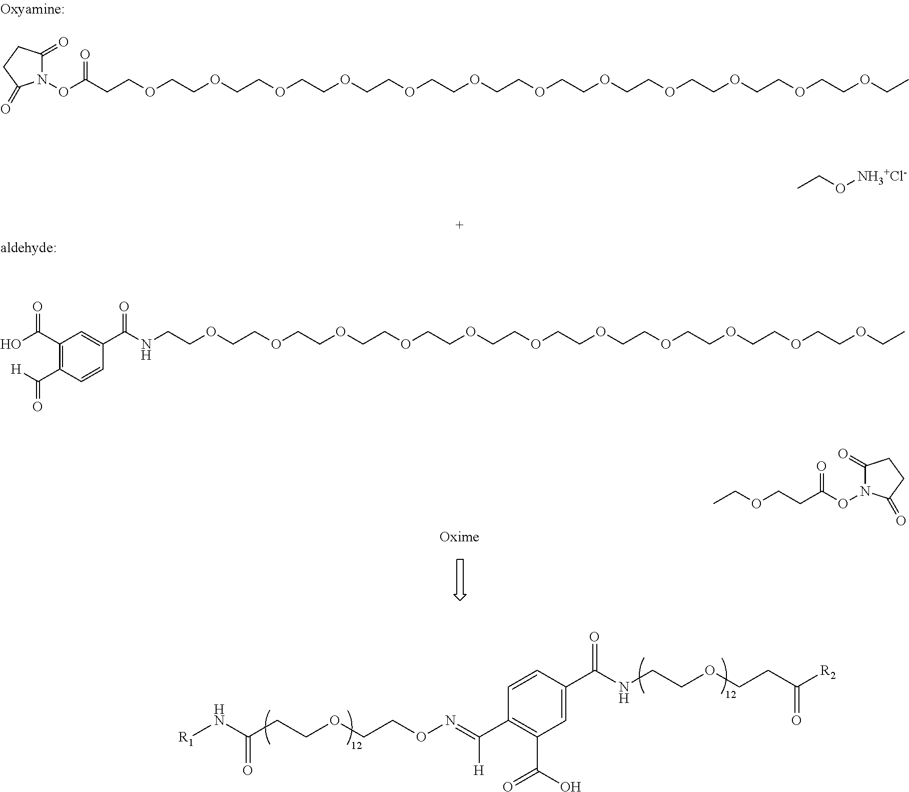

In another aspect, a specific binding pair is azide/acetylide or acetylide/azide. Azide can form a selective linkage with acetylide as shown below.

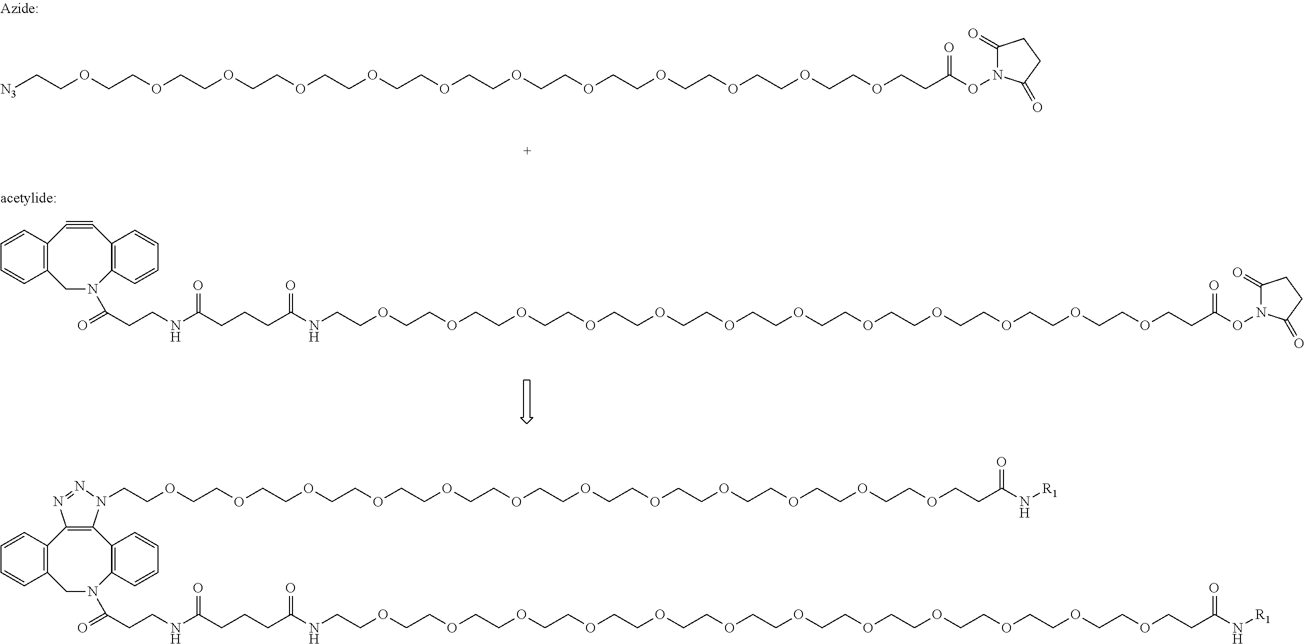

The chemical ligation between biotin/streptavidin, oxyamine/aldehyde or between azide/acetylide occur very rapidly under physiological conditions and none of the reagents involved reacted irreversibly with any biological structures, resulting in rapid and specific linkage of the members of specific binding pairs to the desired cells.

Carriers suitable for practicing the invention can be any physical carrier that facilitates separation of a cell attached to the carrier from the one that are not attached to the carrier. Examples of carriers include, but are not limited to, a magnetic bead or a particle of a given size, weight or density that can be used to identify a particular cell. For example, a particle of a different size can be used to identify the cell to which it is bound by using a technique that sorts based on size (e.g., size exclusion chromatography or a molecular sieve).

Methods of affixing a member of a specific binding pair to a cell are described herein. For example, a tumor cell or dendritic cell can be labeled with biotin. Prior to labeling, tumor cells in T75 flasks or the DC cells in 100 mm-petri dish are washed twice with PBS. The labeling is carried out in T75 flasks for the tumor cells (about 10 million cells/flask) or 100 mm petri dish for the DC cells (about 10 million cells/dish). The tumor cells are then labeled by incubating with 2 μl of NHS-dPEG₂₄-Biotin (25 mg/ml, Quanta Biodesign) in 10 ml of PBS per flask and the DC cells are labeled by incubating with 10 μl of NHS-dPEG₂₄-Biotin in 10 ml of PBS per dish at 4° C. for 40 minutes.

Methods of affixing the complementary member of a specific binding pair to a carrier such as a magnetic bead are generally known in the art. For example, Abe et al. (2008) Journal of Magnetism and Magnetic Materials 321(7):645-9 describes methods of conjugating a bioactive molecule to a magnetic ferrite nanobead for medical applications. Further, a member of a specific binding pair can be conjugated to a carrier via a cleavable linkage that enables cleavage of the specific binding pair from the carrier, as provided below.

Carriers conjugated to a member of a specific binding pair can be added to the cells at any time before separating cells linked to the complementary member of a specific binding pair from the cell mixture. If the carriers are added before cell fusion takes place, the cells can fuse on the surface of the carriers. When the carriers are added during cell fusion or after cell fusion is completed, the carriers can then directly bind the fused cells. After the first separation, carriers conjugated to the member of a specific binding pair can then be added to the cells.

Cleavage of the carrier from a member of a specific binding pair can be accomplished by a number of different approaches. In one aspect, a calmodulin/calmodulin binding protein linkage is used to affix a member of a specific binding pair to a carrier which can then be cleaved by Ca++ ions when needed. In another embodiment of the invention, a disulfide linkage is used to affix a member of a specific binding pair to a carrier and cleavage can occur by the addition of a reducing reagent. In yet another aspect of the invention, an oligonucleotide hybrid linkage is used to affix a member of a specific binding pair to a carrier and can be cleaved by nuclease treatment. For example, an oligo(A) linked to a member of a specific binding pair can be hybridized to an oligo(T) that is linked to a carrier and the oligo(A-T) linkage can be cleaved by a nuclease. Oligo(T) conjugated magnetic beads, for example, are commercially available from Invitrogen (Carlsbad, Calif.). In one embodiment, one of the two oligo strands is a ribonucleotide strand and the cleavage is done by a RNAse. In another embodiment, both oligo strands are ribonucleotide strands that are cleaved by RNAse.

In some embodiments of the invention, at least two cells of different cell type are put into contact with one another, under conditions that promote cell fusion. Such fusion-promoting conditions are well known to the artisan, and typically involve the addition of an agent that promotes cell fusion. These agents are thought to work by a molecular crowding mechanism to concentrate cells to an extent that they are in close enough proximity to cause fusion of cell membranes. While the invention contemplates any agent that meets these characteristics, exemplary useful agents are polymeric compounds, like polyethylene glycols. An effective amount of such an agent generally will be from about 20% to about 80% (w/v). A preferred range is from about 40% to about 60%, with about 50% being more preferred.

Also contemplated herein are fused cells of higher order, which are fusions between more than two cells. In each case, all that is needed is an additional specific binding pair. For example, three different cells are affixed with three different members of a specific binding pair and after they are fused, they can be separated from unfused cells with the complementary member of a specific binding pair. Thus, as used herein, the term “fused cell” contemplates fusions between two or more reactant cells of the same cell type or two or more reactant cells of two or more different cell types. Thus, each of the reactant cells in a fused cell does not have to be different from all other reactant cells. For example, two reactant cells of one cell type can be fused with a reactant cell of another cell type to form a fused cell.

Reactant cells that can be used to generate a fused cell can be any living cell that is desirous to be combined with another cell. For example, a hybrid cell preparation comprises a primary tumor cell and an antigen presenting cell (APC) as reactants. Such hybrid cells may be used as cellular vaccines to induce an immune response against a tumor. The tumor cell may be of any type, including the major cancers, like breast, prostate, ovarian, skin, lung, and the like. The APC preferably is a professional APC, like a macrophage or a dendritic cell. Due to their superior antigen presentation capabilities, dendritic cells are more preferred. Both syngeneic and allogeneic fusions are contemplated herein.

An additional embodiment is a fused cell that comprises a pathogenic cell and an APC. These fused cells also are useful as cellular vaccines. Again, antigen presenting cells, and dendritic cells in particular, are favored. The pathogenic cell, on the other hand, may be of virtually any type. For example, it may be a bacterial cell (Helicobacter, etc.) that has had its cell wall removed. The pathogenic cell may be a fungal cell, like Candida, Cryptococcus, Aspergillus and Alternaria.

The pathogenic cell also may be a parasitic cell from, for example, trypanosomal parasites, amoebic parasites, miscellaneous protozoans, nematodes, trematodes and cestodes. Exemplary genera include: Plasmodium; Leishmania; Trypanosoma; Entamoeba; Naeglaria; Acanthamoeba; Dientamoeba; Toxoplasma; Pneumocystis; Babesia; Isospora; Cryptosporidium; Cyclospora; Giardia; Balantidium; Blastocystis; Microsporidia; Sarcocystis; Wuchereria; Brugia; Onchocerca; Loa; Tetrapetalonema; Mansonella; Dirofilaria; Ascaris (roundworm); Necator (hookworm); Ancylostoma (hookworm); Strongyloides (threadworm); Enterobius (pinworm); Trichuris (whipworm); Trichostrongylus; Capillaria; Trichinella; Anasakis; Pseudoterranova; Dracunculus; Schistosoma; Clonorchis; Paragonimus; Opisthorchis; Fasciola; Metagonimus; Heterophyes; Fasciolopis; Taenia; Hymenolepis; Diphyllobothrium; Spirometra; and Echinococcus.

In another embodiment, the inventive fused cell preparation comprises a target cell against which immune tolerance is desired and an antigen presenting cell that lacks an accessory factor needed for an immunogenic response. Typically these APCs lack B7 (e.g., B7.1 or B7.2); exemplary cells are naïve, immature B cells and fibroblasts, but any cell capable of presenting antigen (having MHC molecules), yet lacking an accessory molecule, will suffice. In the case of B7, specific antibodies are known, and the artisan will be well apprised of methods to ascertain whether any particular cell type lacks B7. Naïve B cells are preferred because they express high levels of MHC molecules and all the adhesive molecules known in the art to be necessary for efficient cell-cell contact.

In any event, the resultant fused cells have the ability to present antigen to the immune system, since they bear class I and class II MHC molecules, yet they will not have the ability to activate the immune system, since they do not have the necessary accessory markers, like B7 (CD28 or FLTA4 ligands). Thus, instead of inducing an immune response, these fused cells will induce apoptotic clearance, thereby rendering the immune system tolerant to the target cell antigens presented by these hybrids. Such immune cell hybrids are useful in treating autoimmune disorders like transplant rejection.

Altogether, the methods described herein enhance the efficiency of separation for a fused cell, so that a sufficient amount of hybrid or other fused cells, for example, can be collected and used for preparing a therapeutic vaccine. Accordingly, the invention in another aspect provides a fused cell or a substantially pure population of fused cells prepared by any of the embodiments of the inventive methods.

Method for Creating a Fused Cell

In addition to devising a more efficient cell sorting/separation method for fused cells, also contemplated in the present invention is a method for enhancing or increasing the rate of cell fusion. As an example, the rate of fusion between dendritic cells and tumor cells is about ˜2%-10%, indicating that only about ˜2%-10% of tumor cells are fused, as observed in prior fusion studies using conventional methods. Also, only about 0.08% of B-cells are fused when mixed with myeloma cells for preparing hybridomas. This low fusion rate makes it extremely difficult to generate a sufficient amount of hybrid tumor/DC cell vaccines for small and early stage tumors. Another example of inefficient cell fusion is between a plasma cell and a cancer cell which is useful in preparing monoclonal antibodies.

The inventors have surprisingly discovered that specific binding pairs can drastically increase the fusion rate between two or more cells when one reactant cell or cells is linked to a member of a specific binding pair and the other reactant cell or cells is linked to the complementary member of the specific binding pair. The resulting fusion rate is so effective that the unfused cells do not need to be removed from the final product.

Accordingly, a method is provided for creating a fused cell that comprises contacting a first cell with a second cell under conditions suitable for the first cell and the second cell to fuse, wherein the first cell is linked on its cell surface to a member of a specific binding pair, and the second cell is linked on its cell surface to the complementary member of a specific binding pair, thereby preparing a cell fused between the first cell and the second cell. Suitable conditions for cell fusion and suitable specific binding pairs are described herein. The method for affixing the specific binding pair members to the reactant cells can also be performed as described above.

In the case of fused cells of higher order, all that is needed is an additional specific binding pair. For example, three different cells are affixed with two different specific binding pairs, e.g., A, A′ and B, B′. A and B are each affixed to a first and second reactant cell, respectively, and A′ and B′, which are the complementary members to the “A” member of a specific binding pair and the “B” member of the specific binding pair, respectively, are both affixed to the third reactant cell. When placed in contact with each other, the three cells bind to one another by means of complementary binding between members of the specific binding pairs.

Isolation of hybrid cells generated by this method from unfused reactant cells can be accomplished by methods known in the art, such as fluorescence-activated cell sorting (FACS), or the methods disclosed supra. For example, each reactant cell can be linked to an additional but different member of a specific binding pair facilitating isolation of the cell with a carrier linked to a complementary member of a specific binding pair.

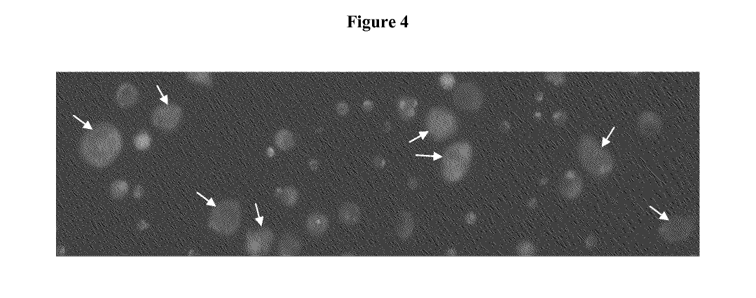

Alternatively, because the fusion rate by this method is so efficient, the unfused cells do not need to be removed from the final product. For example, between the two reactant cell types, dendritic cells are placed in an excess amount (e.g., 5 fold excess) in the cell mixture to optimize the fusion rate with the cells of the other type. The excess unfused dendritic cells do not need to be removed, and actually are beneficial to antigen presentation in the composition. Also see FIG. 4. The cell fusion rate is then considered to be the fusion rate of the cells of the latter cell type. Accordingly, in one aspect, the cell fusion rate is from about 50% to about 100% of total cells. In another aspect, the cell fusion rate is from about 50% to about 60%, from about 60% to about 70%, from about 70% to about 80%, from about 80% to about 90%, or from about 90% to about 100%. In yet another aspect, the cell fusion rate is from about 60% to about 98%, from about 70% to about 95%, or from about 90% to about 95% total cells.

It is also contemplated that the method of the present invention can be used to improve the efficiency of electrofusion. During an electrofusion procedure, a pre-fusion dielectrophoresis is performed to align the cells, followed by a DC pulse to electroporate the cells. The pre-fusion dielectrophoresis step is critical as it organizes the cells in appropriate approximate for cell fusion. With the method of the current invention, the cells are already attached to each other due to the members of a specific binding pair linked to the cells. This attachment can greatly improve the efficiency of the pre-fusion dielectrophoresis or replace it in an electrofusion procedure.

It is further contemplated that a machine or other automated equipment such as a “robot” can be used to carry out the production and isolation of the fused cells. For example, starting from a single cell suspension of a patient's harvested tumor and DCs produced from the patient's apheresis product, tumor/DC cell hybrid cells can be produced simply through a series of robot controlled pipetting operations. Magnetic separation can be accomplished by the robot simply by placing the reaction vessel into and out of a magnetic field before, after or during pipetting.

Kits of the Invention

The present invention also contemplates kits for preparing fused cells. These kits are useful in implementing the inventive method of preparing fused cells. A preparation kit, for example, contains at least one specific binding pair, and instructions for affixing the members of the specific binding pairs to a cell. The inventive fused cell preparation kit may additionally contain one or more carriers, an agent(s) for affixing a member of a specific binding pair to the carrier, and instructions for affixing a member of a specific binding pair to the carrier. Agents that promote cell fusion and instructions to use them, in a further aspect, can also included in the kit. In yet another aspect, the kit further comprises one or more agents for cleaving a member of a specific binding pair from a carrier.

Methods of Treatment

The methods and products described herein are useful in therapeutic and prophylactic treatment methods. Such a method involves administering to a patient a hybrid between a “target” cell and a second, typically antigen-presenting, cell. The “target” cell is one against which an immune response is sought. The immune response may be positive or negative, depending on the disorder to be treated. For example, a positive immune response is desirable in treating cancer or parasitic diseases, but a negative immune response is desirable in preventing transplant rejection.

An exemplary cancer treatment method involves (a) isolating a tumor cell and a dendritic cell from a patient; (b) preparing a tumor cell/dentritic cell fusion by the method of any of the embodiments of the invention; and (c) administering the hybrid cell to the subject, thereby treating the subject. The hybrid cell is isolated and administered to a patient in an acceptable excipient. In order to avoid administration of viable cancer cells, it is contemplated that the tumor cells be treated so that they do not pose a risk to the patient. For example, the tumor reactant cells can be irradiated prior to cell fusion. This step renders the cell unable to divide but does not prevent efficient presentation of the tumor antigen(s) by the resultant hybrid cell. Both syngeneic and allogeneic fusions are contemplated.

Also contemplated in this invention is exposing the fused cells to an adjuvant, cytokine or other agent (such as one that can activate the toll like receptor) that would otherwise be co-administered or sequentially administered in the course of treatment and avoid the harmful side effects of the adjuvant, cytokine or other agent.

The cancer treatment, however, may also optionally supplemented with traditional cancer therapy. For example, the use of additional antineoplastic agents in conjunction with the fused cells is contemplated herein. One class of such agents is immunomodulators. These include cytokines and lymphokines, especially interleukin-2 (IL-2) and IL-2 derivatives, like aldesleukin (Proleukin, Chiron Corp.). The use of IL-2 is preferred because it should further enhance the immune response generated by the hybrid cell. As used herein, “interleukin-2” is used generically to refer to the native molecules and any derivatives or analogs that retain essential interleukin-2 activity, like promoting T cell growth. Other lymphokines and cytokines may also be used as an adjunct to treatment. Examples include interferon gamma (IFN-γ), granulocyte macrophage colony simulating factor (GM-C SF), and the like.

The present invention can be used to treat any disorder associated with a pathogenic organism. In this modification, the reactant cells will be APCs and cells isolated from the pathogenic organism. Otherwise, the treatment would be accomplished as in cancer treatment.

A different aspect of the invention comprehends a method of treating autoimmune disorders. The method is accomplished in essentially the same manner as the cancer treatment set out above. The primary difference being the identity of the reactant cells. In the case of autoimmune disorders, the goal is to diminish or eliminate an immune response, whereas in cancer treatment the goal is to create or enhance an immune response.

The ability to use the inventive hybrids in treating autoimmune disorders derives in part from the observation that certain cells can present antigen, yet they lack the accessory molecules to provide a positive immune response. Typically these cells lack B7, and they may be immature B cells or fibroblasts, for example. In fact, antigen presentation by such cells generates a negative immune response. It tolerizes the immune system, inducing apoptosis of specific antigen-reactive immune cells.

Thus, the method of treating autoimmune disorders utilizes an APC, deficient in an accessory interaction, and a “normal cell” as the reactants. The “normal cell” is any target cell to which immune tolerance is desired. It may be from a transplant organ, for example, in a method of preventing transplant rejection. In the case of treating or preventing diabetes, by transplantation or otherwise, on the other hand, the normal cell may be an Islet cell. Such a method can be adapted to tolerize the immune system against any type of cell.

The Examples are for the purpose of illustration only and are not intended to limit the scope of the invention.

### EXAMPLES

### Example 1

### Materials and Methods for Preparing and Using Poly(U) Magnetic Beads

### Poly(U) Magnetic Beads

1. Prepare immediately before use a solution of 10 mg/ml of sodium periodate (also known as “sodium meta periodate”) and cover to protect from light.

2. Add 2.5 ml of the 10 mg/ml sodium periodate solution to 25 mg polyribo(U) (Midland Certified Chemical Co.) directly into the vial it is packaged in. Cover with aluminum foil and let stand in dark for 30 minutes.

3. While the poly(U) is oxidizing, wash 2 ml of 270 amine polystyrene magnetic beads from Dynal in PBS three times to remove any azide or other material from supplier and prepare and wash a 10 ml Zeba Desalt spin column from Thermo Scientific (Pierce) with PBS so that the oxidized Poly(U) that comes through the spin column ends up in PBS.

4. Remove all periodate from the poly(U) by passage through a spin column, add the 2.5 ml of oxidized poly(U) to the PBS treated (solvent free) beads in a small vial, and then add 25 μl of 5M sodium cyanoborohydride (Pierce) dissolved in 1 M NaOH (make solution immediately before use) and agitate to keep beads in suspension. React for 2 hours with agitation.

5. Remove the reaction solution via magnetic separation, thoroughly wash beads in PBS and then transfer into 1.5 mls of 0.1M KCO₃ (potassium carbonate) pH 10.2, then add 80 mg of solid succinic anhydride (Aldrich) and agitate solution to keep beads suspended for 30 minutes. Use magnetic separation to remove beads from the anhydride and carbonate and wash four times into PBS. Keep polyribo(U) beads at 4° C. When reacting the beads with the anhydride in carbonate, CO₂ will be produced and therefore the reaction should be run in a 2 ml ependorf tube tightly sealed with parafilm to keep the lid from popping off.

Poly(U)-Streptavidin Beads

1. Take 100 μl Poly(U) beads and wash with PBS 4 times, re-suspend in 100 μl PBS.

2. Add 10 μl Oligo₁₅(A)Biotin(5′) (200 μM), mix, incubate at room temperature for 15 minutes and then vortex.

3. Wash with PBS four times, then re-suspend in 100 μl PBS.

4. Add 5 μg streptavidin, mix, and then incubate at room temperature for 15 min, then vortex.

5. Wash with PBS three times, re-suspend in 100 μl PBS. For longer storage, re-suspend the beads in PBS containing 0.01% sodium azide.

The cells should be placed on ice before adding to the beads and binding on ice. It has been observed that 100 μl beads can bind and isolate up to 2 million cells. For cleaving poly(A) from the beads, 1 μl RNase A (100 mg/ml) is needed for 10 μl beads and the treatment time for RNase A treatment is about 15-30 min.

Poly(U)-Oxyamine Beads

1. Mix 100u1 OligoA-NHS ester-PEG-Oxyamine-Phylamid(3′):

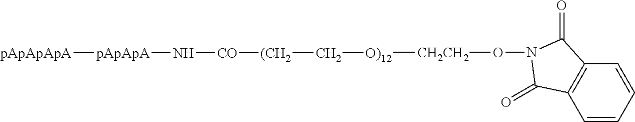

with 100 μl Hydrozine hydrate soluation (sigma) and incubate at room temperature for 2 hours.

2. Pass through Spin Column twice, 1000 g×2 min each.

3. Use the elution (200 μl) to resuspend the pellet of 200 μl Poly(U) beads and incubate at room temperature for 30 min.

4. Wash with PBS three times and resuspend the beads in 200 μl PBS.

5. The beads are ready and are good for 2 days at 4° C.

For 1×10⁶ aldehyded cells, a minimum of 100 μl beads is needed. Further, before the binding reaction, the aldehyded cells need to be incubated on ice for at lest five min. The binding reaction needs to take place on ice for 15-30 min. Finally, only freshly aldehyded cell can be used.

### Bead Purification

1. Biotin label two T75 flasks of B16. Aldhyde label two T75 flasks of B16 cells.

2. Stain two of the biotinylated flasks green; stain the two aldhyde flasks red.

3. Irradiate all the cells.

4. Fuse the two types of cells with virus envelope.

5. Add 2 ml ice cold fusion mixture (5×10⁶ cells/ml) to the pellet of 100 μl Poly(U)-SA beads, mix, and incubate on ice for 15 min.

6. Wash the cell/beads 3 times with PBS and resuspend them in 100 μl PBS.

7. Add 10 μl of RNase A (100 mg/ml) and incubate at room temperature for 15 min.

8. Harvest the cells and wash the cells 3 times with PBS by spinning

9. Resuspend the cells in 1 ml PBS.

10. Add the cell suspension to the pellet of 200 μl Poly(U)-Oxyamine beads, mix, and incubate at room temperature for 20 min.

11. Wash the cell/beads mixture 3 times with PBS and resuspend them in 100 μl PBS.

12. Add 10 μl RNase A and incubate at room temperature for 15 min.

13. Check the released cells under fluorescent microscope.

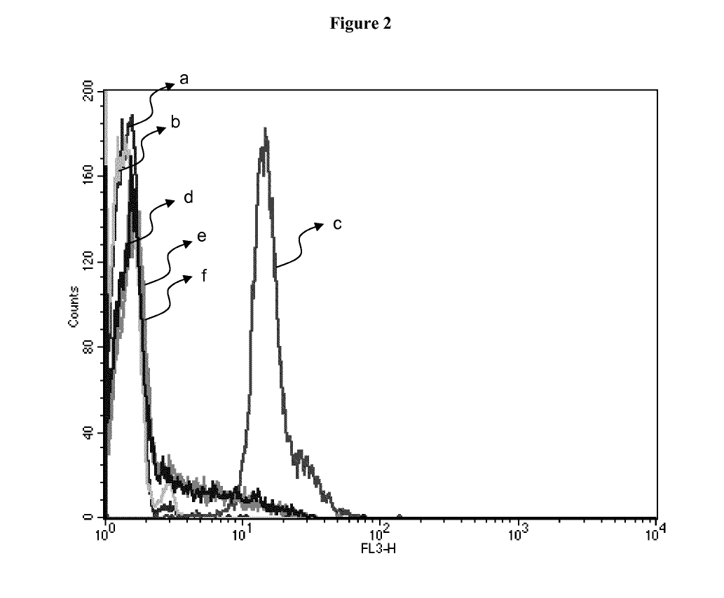

The binding and cleavage between a poly(U) bead and oligo(A) linked specific binding pairs are demonstrated with FASC (fluorescence-activated cell sorting). In FIG. 2, curve (a) shows poly(U) beads, (b) shows poly(U) beads stained with 1 μl streptavidin-PerCP (peridinin chlorophyll protein complex) (0.2 μg/μl), (c) shows poly(U) beads annealed with Oligo(A)-Biotin and stained with 1 μl streptavidin-PerCP, (d) shows poly(U) beads annealed with Oligo(A)-Biotin and stained with 1 μl streptavidin-PerCP and then treated with Rnase A for 10 min, (d) shows poly(U) beads annealed with OligoA-Biotin and stained with 1 μl streptavidin-PerCP and then treated with Rnase A for 20 min, and (e) shows poly(U) beads annealed with OligoA-Biotin and stained with 1 μl streptavidin-PerCP and then treated with RnaseA for 30 min. Curve (c)'s separation from (a) and (b) indicates the binding between the poly(U) beads and the oligo(A) linked biotin/SA. Overlapping between (c), (d), or (e) and (a) then indicates dissociation of the binding that formed in (c).

### Example 2

### Isolation of Dendritic Cell/Tumor Cell Hybrid Cells

This example demonstrates the preparation of a hybrid cell, which is a fused cell created by fusion of a cancer cell and a dendritic cell. These hybrid cells are used as a therapeutic vaccine to prevent cancer in a murine metastatic cancer model system.

### Example 3

### Improved Fusion Rate between Dendritic Cells and Tumor Cells

This example demonstrates the fusion between B16 (mouse melanoma cell line) cells and dendritic cells (DCs) using a biotin-streptavidin(SA)-biotin bridge. In a biotin-SA-biotin bridge, a biotin can be considered as a member of a specific binding pair, and a biotin-SA can be considered as the complementary member of the specific binding pair, for the purpose of this application.

First, B16 and DC cells were labeled with biotin. Prior to labeling, B16 cells in T75 flasks and DC cells in 100 mm-petri dish were washed twice with PBS. The labeling was carried out in T75 flasks for B16 (about 10 million cells/flask) and 100 mm petri dish for DC cells (about 10 million cells/dish). B16 cells were then labeled with 2 μl of NHS-dPEG₂₄-Biotin (25 mg/ml, Quanta Biodesign) in 10 ml of PBS per flask and DC cells were labeled with 10 μl of NHS-dPEG₂₄-Biotin in 10 ml of PBS per dish at 4° C. for 40 minutes.

After biotinylation, cells were collected and washed twice with PBS. The biotinylated B16 cells were stained red with PKH26 (Sigma) and DCs stained with PKH67 (Sigma) green dye according to manufacture's instruction. After dye labeling and washing, DCs were resuspended in PBS. The red dye labeled B16 cells were irradiated at 100 grays, washed once with PBS and resuspended in 5 ml of PBS. One mg of purified streptavidin per 10 million cells was added into the B16 solution and incubated at 4° C. for 20 minutes with occasional gentle mix by shaking. The B16 cells were washed twice with PBS to wash off the excess streptavidin and then resuspended in PBS. Next, DCs and B16 cells were mixed at a ratio of 5:1 in PBS and incubated for 30 minutes at room temperature for biotin-streptavidin binding on the cells to occur. Finally, 0.7 ml of the DC-B16 mixture was aliquoted into each of the 4 mm gap electroporation cuvette (BTX model ECM830) and subjected to electrofusion (450V 60 μs×2 with 200 ms intervals) using 4 mm BTX cuvette. The fused cells were collected and placed in T75 flasks with fresh DC medium and cultured overnight.

In addition to electrofusion, PEG (polyethylene glycol) fusion can also be used to generate hybrid cells between the B16 cells and DC cells. Methods of generating fusion cells are generally known in the art, see, for example, Kohler and Milstein (1975) Nature 256:495-7, Galfre et al. (1977) Nature 266:550-2, Margulies (2005) J. Immunol. 174:2451-2 and Shu et al. (2006) Cancer Metastasis Rev. 25:233-42.

As shown in FIG. 4, DC cells are stained with Green tracker dyes (small circles), tumor cells are stained with Red tracker dyes (medium size circles) and the hybrids are the larger yellowish orange cells (indicated by arrows).

### Example 4

### Mouse Study

Studies carried out in several mouse tumor models can be undertaken in order to determine both the vaccine's therapeutic efficacy and the mass of tumor required to generate sufficient vaccine for effective treatment. Efficacy can be determined by vaccinating mice at defined time intervals following inoculation of test mice with the four murine tumor cell lines, murine melanoma (B16F0), murine leukemia (C1498), murine lymphoma (EL-4) and murine sarcoma (S180). In each model both a local subcutaneous model and a metastatic model can be used. Efficacy can be determined either by tumor size or by counting specific organ metastases. In addition the minimal number of cells required in each model to generate an effective vaccine can be determined.

Generation of a therapeutic CD8 cellular immune response is dependent upon more than MHC class I presentation of “foreign” epitopes generated from proteins residing within the cytoplasm of the dendritic cell. The milieu in which the presenting dendritic cell resides and the cytokine micro-environment at the site of antigen presentation are critical to CTL generation from presented “tumor” epitopes. In its role as the control center of the immune system the dendritic cell must choose between tolerance and rejection whenever aberrant proteinaceous material is encountered. In the absence of appropriate “danger” signals the DC triggers the production of CD4 regulatory T-cells and CD8 suppressor T-cells which blunt the CTL immune response. The choice for rejection is made when the aberrant material is encountered within an “inflammatory” environment as is the case at the site of a bacterial infection, stimulating strong CTL production. This aspect of the dendritic cell response explains the remarkable success of Coley's Toxin over 100 years ago in mounting strong and effective immune responses against tumors. It is crucial that once tumor/DC cell hybrids can be efficiently produced that they be administered to the patient within an appropriately “inflammatory” environment. Doing so in present day medicine does not require eliciting a bacterial infection but can be accomplished by providing the appropriate cytokines and toll like receptor (tlr) ligands at the site of therapeutic hybrid cellular vaccination. While this is usually accomplished by co-injection of a vaccine together with such cytokines and/or tlr ligands, we suspect that the hybrid tumor/DC cell vaccine together with our improved means of its preparation may offer an alternative approach. The logical reason for providing the appropriate cytokines and tlr ligands at the tissue site of vaccination is so that the appropriate micro-environment is present at the time of dendritic cell/tumor antigen recognition, but in the case of our hybrid cell vaccine the time of “recognition” is during or after fusion of the DC and tumor cell during the vaccine production process. Preliminary studies suggest, but must be further confirmed, that adding a simple step in the vaccine production process could result in providing the same “microenvironment” effect to ensure strong CD8 CTL production and to limit any CD4 regulatory T-cell or CD8 suppressor T-cell generation by the vaccine which would counteract the desired anti-tumor effect. The last step in the proposed production process of the hybrid cell vaccine, after the DC has already fully encountered the tumor cell “antigen” and is still attached to SPM MBs, offers a unique opportunity to expose the hybrid cell to cytokines and tlr ligands. By exposing the hybrid cells to an idealized microenvironment to elicit a strong CTL response and then, after so “activating” the hybrid cells, removing these agents that have known adverse side effects in patients by simple magnetic separation/washing the most effective cytokine/tlr ligand “cocktail” can be used without exposing the patient to it. One of the best indications of appropriately activated DCs for strong CTL stimulation is the production of IL-12. We will evaluate this unique approach to hybrid cell maturation/activation by determining if the addition of such a simple step in the production process results in sustained IL-12 production by the hybrid cell product. The cytokine/tlr ligand “cocktail” we will first study will include IL-12, IL-18 and CpG DNA.

### Example 4

### Clinical Trial

Fifteen patients diagnosed with Stage III or IV malignant melanoma will be considered for this study. Written informed consent will be obtained from all subjects before any study related procedures (including any pretreatment procedures or evaluations) are performed. A minimum of 1.25 million hybrid cells will be produced for each patient before he/she is enrolled in this protocol and may receive the first vaccination (one million of the hybrid cells alliquoted in four doses for administration to the patient; 250,000 are used for lot release quality control testing). After the initial vaccination, the patient will be revaccinated every four weeks, depending on eligibility. A dose of 250,000 hybrid cells (range of 200,000-300,000) will be administered during each vaccination. Each patient may receive up to four vaccinations. The hybrid cell dose will thawed and diluted to a final volume of 1 ml with Sterile Saline for Injection containing 5% human serum albumin. The hybrid cell dose is to be injected subcutaneously into an area of lymph nodes in the axilla or inguinal area of the patient, using a 23 gauge or larger needle. This site will be rotated to avoid injection in the same lymph node bed on two consecutive administrations. As one of the earliest measures of hybrid cell vaccine efficacy will be a determination of the increase in gamma interferon secreting CD8 T-cells. PBMCs for this purpose will be obtained by separate 20 ml blood draws from the patient taken 3 weeks after each vaccination. The PBMCs will be isolated from the blood by density gradient centrifugation and cryopreserved. Prestudy PBMCs will be obtained from the apheresis product obtained during hybrid cell production procedures.

All PBMCs from one patient will analyzed at the same time, after completion of the vaccine therapy, for the number of IFN-γ expressing T cells following in vitro stimulation with autologous tumor cell lysate, using Becton Dickinson's Fastlmmune intracellular IFN-γ staining kit. Standard follow-up parameters will be measured for each patient. Disease assessments will be done Months 3, 6, 9, 12 and 18 using CT and a PE, CB, CMP and Sed rate will be done at the same time intervals along with weight and vital signs. After the 18 month follow-up period, the patient will have completed this protocol. Overall survival of the patients will be captured by the facilities Cancer Registry.

1. A method for separating a fused cell from a population of unfused cells comprising:
(a) contacting a first cell or cells with a second cell or cells under conditions suitable for cell fusion, wherein the first cell or cells is/are linked to a member of a first specific binding pair and the second cell or cells is/are linked to a member of a second specific binding pair, and wherein the member of the first specific binding pair and the member of the second specific binding pair are different;
(b) adding a carrier conjugated to a member of a specific binding pair complementary to the member of the first specific binding pair;
(c) isolating cells linked to the member of the first specific binding pair based on properties of the carrier;
(d) adding to the cells isolated in (c) a carrier conjugated to a member of a specific binding pair complementary to the member of the second specific binding pair;
(e) isolating cells linked to the member of the second specific binding pair based on properties of the carrier, wherein the fused cells are separated from the unfused cells.

2. The method of claim 1, wherein the fused cell comprises a cell of a first cell type and a cell of a second cell type.

3. The method of claim 2, wherein the first cell type is a dendritic cell and the second cell type is a tumor cell.

4. The method of claim 1, wherein the first cell or cells is/are linked on the cell surface to the member of the first specific binding pair.

5. The method of claim 1 or 4, wherein the second cell or cells is/are linked on the cell surface to the member of the second specific binding pair.

6. A method for enhancing the rate of cell fusion between reactant cells comprising contacting a first reactant cell or cells and a second reactant cell or cells under conditions for cell fusion, wherein the first reactant cell or cells is/are linked to a member of a specific binding pair and the second reactant cell or cells is/are linked to a complementary member of the specific binding pair.

7. The method of claim 6, wherein the first reactant cell or cells is/are of a first cell type and the second reactant cell or cells is/are of a second cell type.

8. The method of claim 7, wherein the first cell type is a dendritic cell and the second cell type is a tumor cell.

9. The method of claim 6, wherein the first reactant cell or cells is/are linked on the cell surface to the member of the specific binding pair.

10. The method of claim 6 or 9, wherein the second reactant cell or cells is/are linked on the cell surface to the member of a specific binding pair complementary to the member of the specific binding pair.

11. The method of claim 1 or 6, wherein the carrier is a magnetic bead.

12. The method of claim 1, wherein the carrier is conjugated to a member of a specific binding pair via a pair of complementary oligonucleotide, a calmodulin/calmodulin binding protein linkage, or a disulfide linkage.

13. The method of claim 1 or 2, wherein the specific binding pairs each is selected from the group consisting of biotin and streptavidin, oxyamine and aldehyde, azide and acetylide.

14. An isolated hybrid cell prepared by the method of claim 1 or 6.

15. A substantially pure population of fusion cells prepared by the method of claim 1 or 6.

16. A kit for separating a fused cell from a population of unfused cells comprising a first specific binding pair, a second specific binding pair and one or more carriers.

17. A method for treating a tumor in a subject, comprising:
(a) isolating a tumor cell and a dendritic cell from the subject;
(b) preparing a hybrid cell with the method of claim 1 or 6; and
(c) administering the hybrid cell to the subject, thereby treating the subject.
